(19) United States
(12) Patent Application Publication
(10) Pub. No.: US 2024/0415687 A1
(43) Pub. Date: Dec. 19, 2024

(54) METHOD FOR FASTENING AN ORTHOSIS, AND ORTHOSIS

(71) Applicant: OTTOBOCK SE & CO. KGAA, Duderstadt (DE)

(72) Inventors: Matthias VOLLBRECHT, Duderstadt (DE); Laura HOFFSÜMMER, Duderstadt (DE)

(21) Appl. No.: 18/706,598

(22) PCT Filed: Nov. 10, 2022
(86) PCT No.: PCT/EP2022/081529

§ 371 Date: May 1, 2024

(30) Foreign Application Priority Data
Nov. 10, 2021 (DE) 10 2021 129 271.9

(51) Int. Cl. A61F 5/01

(52) U.S. Cl. CPC A61F 5/013

(57) ABSTRACT

The invention relates to a method for fastening an orthesis to an upper arm, the orthosis comprising a dimensionally stable shell (2) with a ventral tab (6) and a dorsal tab (8), wherein the ventral tab (6) and the dorsal tab (8) are or can be connected by means of a tensioning element, the method comprising the following steps: applying the dimensionally stable shell (2) to an upper arm and tensioning the tensioning element so that a tensile force is exerted on the ventral tab (6) and the dorsal tab (8), said force acting in the proximal direction.

The invention relates to a method for fastening an orthesis to an upper arm, the orthosis comprising a dimensionally stable shell with a ventral tab and a dorsal tab, wherein the ventral tab and the dorsal tab are or can be connected by means of a tensioning element. The invention also relates to an orthosis for carrying out such a method.

Orthoses are orthopedic devices by way of which a body part of a wearer of the orthosis should be protected, supported and/or strengthened. The present invention relates to orthoses for the upper limb, which are arranged in the proximal area of the upper arm and on the wearer's shoulder. They are used, for example, for patients who have had a stroke or in the case of plexus palsy. Such orthoses are particularly advantageous if the wearer's upper arm muscles are not strong enough to hold the wearer's arm on the shoulder joint. The orthoses should reduce the subluxation in the shoulder joint and absorb the force, in particular the weight of the respective arm, and transfer it to the shoulder and the opposite, contralateral side. Such orthoses may be referred to as shoulder orthoses and are also used, for example, to stabilize the shoulder following an injury or after an operation performed on a shoulder, whilst still enabling and possibly supporting at least a restricted movement of the shoulder. In doing so, an attempt is made to protect the weakened shoulder in an optimum position and at the same time to enable or facilitate an at least restricted movement.

To this end, conventional shoulder orthoses have a base body made of an elastic material. Said base body comprises, for example, a shoulder section which, when mounted, rests on the shoulder of the person wearing the shoulder orthosis. An arm section, normally tube-shaped, is located on said shoulder section, the former being arranged around the upper arm of the person wearing the shoulder orthosis. Since the base body is made of an elastic material, the shoulder orthosis can be pulled over the arm like a shirt sleeve until it rests on the shoulder. Using the at least one strap provided, which is placed around the upper body of the wearer of the orthosis, the base body of the shoulder orthosis is held in position and at the same time, in many designs, a tensile force is exerted on at least the shoulder area of the base body. In this case, the at least one strap can be connected at at least one point to the base body in a detachable manner in order to make it is easier to apply the orthosis.

When a shoulder orthosis from the prior art is applied, the arm of the wearer of the shoulder orthosis is first introduced into the arm section of the base body and the base body pulled upwards along the arm until it reaches its final position at the shoulder. Via the one strap which, for example, is connected at one of its ends to the base body of the shoulder orthosis such that it cannot be detached, a tensile force can now be applied to the base body, which is applied by way of the base body to the shoulder on which it rests. Advantageously there is a fastening element at the free end of the at least one strap, which may be part of a velcro fastener. The corresponding counter-piece is on the base body of the shoulder orthosis, in particular on the shoulder section of said base body. This renders it possible to individually adjust the effective length of the strap and therefore also the tensile force exerted by the strap to the wearer of the shoulder orthosis. The wearer of the shoulder orthosis or someone providing assistance consequently exerts a tension on the at least one strap before connecting the free end of the strap to the base body of the shoulder orthosis. A tensile force is thus exerted on the base body of the shoulder orthosis, where it is exerted on the shoulder section of the shoulder orthosis in particular. However, since said base body is made of an elastic material, it yields to said tensile forces, at least over a certain area.

The base body of the shoulder orthosis is usually arranged directly on the skin of the person wearing the shoulder orthosis. The inside of the base body may be coated in order to guarantee the best possible adhesion of the base body of the shoulder orthosis to the wearer's skin. This prevents, or at least renders more difficult, a slipping of the shoulder orthosis while the shoulder is moving. The disadvantage is that when the strap is connected to the base body of the shoulder orthosis, a tensile force is exerted on this base body which causes it to deform. Due to the increased friction between the skin of the wearer of the orthosis and the base body, this deformation is transferred to the patient's skin and thus to the actual shoulder element to be supported. Closing such a shoulder orthosis consequently leads to a movement of the shoulder, which is usually pulled forward. The shoulder is then supported and fixed in this position by the shoulder orthosis. However, this position is not the desired, optimum, healthy position, but a displaced position in relation thereto. As a result, it is disadvantageous to support and fix the shoulder in this position.

The same problem arises if the strap is not connected at one end to the base body of the shoulder orthosis in a detachable manner, but is connected at both ends to the base body such that it cannot be detached, for example by sewing it on. The strap can also be designed as a single piece with the base body, such that the latter is also made of an elastic material. The base body is still deformed and twisted when such a shoulder orthosis is applied, meaning that the significant friction between the base body and the skin of the wearer of the shoulder orthosis again causes a movement and displacement of the shoulder joint, so that there is also no guarantee in this case that the shoulder will be supported in an optimum position.

DE 10 2011 119 397 A1 therefore discloses a shoulder orthosis in which straps, which transfer the force to the base body of the orthosis, are made of one and elastic material. This prevents the straps and the base body from deforming, at least in the area that is connected to the straps; however, the force that acts when the orthosis is tensioned will still cause the upper arm to be deformed and displaced relative to the wearer's shoulder.

The invention is therefore based on the task of eliminating or at least reducing the disadvantages of the prior art.

The invention also solves the addressed task by way of a method for fastening an orthosis of the type described here, which is characterized by the following steps:
applying the dimensionally stable shell to an upper arm,
tensioning the tensioning element so that a tensile force is exerted on the ventral tab and the dorsal tab, said force acting in the proximal direction.

The dimensionally stable shell of the orthosis is preferably designed so that its geometric shape is adapted to the upper arm. This can be achieved with a shell that is individually adapted to the respective wearer's upper arm or with a generic shell that is generally adapted to an upper arm. Preferably, the dimensionally stable shell is not positioned directly on the wearer's skin, which may lead to pain or at least an unpleasant pressing sensation, especially during movement of the upper arm, due to the dimensional stability. This is especially likely when the dimensionally stable shell is not individually adapted to the upper arm.

The dimensionally stable shell preferably has a padding element on a side facing the upper arm. The padding element constitutes part of the shell and is arranged on the dimensionally stable shell in such a way that it comes into contact with the upper arm when the shell is applied to the upper arm. The padding element is preferably designed to be flexible, particularly preferably it is elastic. In one configuration of the invention, the padding element is designed as a textile padding element.

Consequently, such a padding element, particularly a textile padding element, is itself not necessarily dimensionally stable. In this case, the dimensional stability of the dimensionally stable shell is not caused by the padding element, but by another dimensionally stable component of the shell. The padding element, especially a textile padding element, can be designed to be significantly larger than the dimensionally stable component of the dimensionally stable shell. This means that the padding element comes into contact with a significantly larger area of the arm than the dimensionally stable component of the dimensionally stable shell would.

The dimensionally stable shell is preferably shaped such that it at least partially surrounds the upper arm. It can preferably be closed via a strap or a similar element so that it completely surrounds the upper arm. In this case, the strap or similar element constitutes part of the dimensionally stable shell. For example, such a strap can be fastened at one end to the dimensionally stable shell, preferably in a non-detachable manner, especially preferably by being sewn on, and comprise a form-fitting element at the opposite end, which is configured to engage with a correspondingly designed second form-fitting element fastened, for example, to the dimensionally stable shell. Once such a strap is closed, the wearer's upper arm is fully enclosed by the dimensionally stable shell. Preferably, there is also at least a frictional connection between the dimensionally stable shell, preferably the padding element, and the upper arm so that a force acting on the shell is transferred to the upper arm. The padding element of the dimensionally stable shell is preferably made of an elastic material, which is preferably tensioned when the orthosis is applied to the upper arm. This strengthens the frictional connection between the shell and the upper arm. The dimensionally stable shell, preferably the padding element, is preferably provided with an anti-slip coating, such as a silicone coating, on the side facing the upper arm.

After the dimensionally stable shell has been applied to the upper arm, the dorsal tab and the ventral tab each extend in the proximal direction, i.e. upwards if the upper arm is hanging down. They are or can be connected to each other above the shoulder. In this case, a force acting in the proximal direction does not already need to be transferred to the upper arm. Preferably, this does not occur until the tensioning element is tensioned. However, due to the special design of said tensioning element, no one-sided force is exerted on the element of the orthosis resting on the upper arm, contrary to orthoses from the prior art. Rather, the tensile force is exerted on the ventral tab as well as the dorsal tab. This results in an even force transmission and thus only in a force acting in the proximal direction.

Straps are known from the prior art, for example, which at one end are fastened in a non-detachable manner, for example sewn on, to one of the two tabs, for example the dorsal tab. There is a velcro fastening element at the opposite end, which can engage with a correspondingly designed velcro fastening element, which is preferably located on the other tab, for example on the ventral tab. To tension such a tensioning element, a tensile force is exerted on the strap that only acts on the dorsal tab. Only once the two velcro fastening elements engage and a form-fitting connection occurs is a force exerted on the respective other tab, i.e. in this case the ventral tab. As such, there is no simultaneous application of force on the two tabs during tensioning. This is different in the present invention. In this case, the tensile force is already applied to both the ventral tab and the dorsal tab during tensioning of the tensioning element.

Preferably, the ventral tab and the dorsal tab are connected to each other, particularly preferably fastened to each other, before the tensioning element is tensioned. For example, this may be done using form-fitting elements that are arranged on the two tabs and can interact with one another. In an especially simple configuration, a velcro fastening element is arranged on one of the two tabs and the corresponding velcro fastener counter-element on the respective other tab. Alternatively, a connection element, such as a fabric strip or a textile strip, a belt or a strap, can be used which is or can be connected to the two tabs in order to connect the two tabs to each other. It is important that this can be done either completely or almost completely in the absence of an acting tensile force and can therefore only serve to keep the two tabs in the desired position. The force acting in the proximal direction, which is important for ensuring that the orthosis fits and functions well, is preferably not generated when the two tabs are connected, but is only produced due to the tensioning of the tensioning element.

Particularly preferably, the ventral tab and the dorsal tab are connected to each other, particularly preferably fastened to each other, above the shoulder. This, however, is not essential. A fastening or connection can also occur at any other point.

The invention also solves the task addressed by way of an orthosis for carrying out a method of the type described above, said orthosis comprising a dimensionally stable shell with a ventral tab and a dorsal tab, wherein the orthosis is characterized in that it has a tensioning element by means of which a tensile force can be applied to the ventral tab and the dorsal tab, wherein said tensile force can be increased by tensioning the tensioning element. As previously explained, when increasing the tensile force by tensioning the tensioning element, it is important that the tensile force acts on the two tabs at the same time and is increased accordingly.

A dimensionally stable shell is, for example, a shell that keeps its shape without interference from external forces, with the exception of gravity. If a dimensionally stable shell is placed on a surface, for example, it at least largely, but preferably entirely, retains it shape. A shell made of a simple textile, however, would collapse. The dimensionally stable shell has, at least in sections, a tubular or partially tubular area. A tubular area completely surrounds the wearer's upper arm, i.e. across its entire circumference. A partially tubular section only surrounds the upper arm over part of its circumference. The tubular or partially tubular area of the shell is designed in such a way that it retains its open shape, even in the applied state. The dimensionally stable shell, in particular the tubular or partially tubular area, is preferably designed so that the user can thread it as easily as possible onto the arm treated with the orthosis using their healthy arm until it reaches the desired end position on the upper arm. In a particular configuration, the shell is made to be so dimensionally stable that the shell reduces and/or damps a swinging of the upper arm to which it is applied. Particularly preferably, it suppresses an overshooting. The swinging is preferably reduced by 25%-50%. As a result the arm movement is adapted to a natural movement, in particular to the natural gait when walking (swinging the arms).

The geometric form of the shell is preferably adapted to the upper arm to which the dimensionally stable shell is to be applied. In particular, the dimensionally stable shell is configured to be able to absorb shear forces that are introduced transversely to the thickness of the material. These shear forces occur, for example, when the orthosis is moved against the friction of the skin into the end position while being applied to the upper arm. The dimensionally stable shell preferably does not deform in the process. When the orthosis is being applied to the upper arm, friction with the skin on the wearer's arm occurs in particular if the orthosis has an area that completely surrounds the upper arm and the orthosis is threaded onto the arm. This often results in close contact between the orthosis and the wearer's skin in the lower arm area and then in the upper arm area. A purely textile orthosis would become creased as a result and could not be moved into the end position. This would only be possible by applying a tensile force to the upper end of the textile.

Due to the dimensionally stable property of being able to absorb shear forces, the orthosis can be slid onto the arm when, for example, the tubular or partially tubular area of the shell is moved.

The dimensionally stable shell can be made of a plastic, for example, a fiber-reinforced plastic, such as GRP or CFRP, or a metal, such as aluminium. The dimensionally stable shell preferably has a dimensionally stable foam element. The foam element can be connected to the padding element on the side facing the wearer's skin. In a preferred design, the foam element is connected to a textile on the side facing the wearer's skin. The textile should ensure comfort during wearing. A further layer made of a plastic or a textile may be arranged on the opposite side of the foam element in order to reduce susceptibility to external influences, such as contaminants. The foam element preferably has a flexural modulus of at least 7 MPa, preferably at least 10 MPa, especially preferably at least 13 MPa and at most 100 MPa.

The dorsal tab preferably extends dorsally to just below the shoulder bone (spina scapulae). The distance to the shoulder bone is preferably less than 5 cm, especially preferably less than 2 cm. The dorsal projection forms the counter-support to the ventral flap on the upper arm, which extends ventrally. This course of the straps limits a possible swinging movement of the upper arm backwards and positions the upper arm. The ventral tab of the shell preferably extends on the shoulder girdle to just before the collarbone. The distance to the collarbone is preferably less than 5 cm, especially preferably less than 2 cm. Overall the ventral tab serves to raise the arm when the tensioning element is tensioned. The brace can be individually adapted to the anatomical conditions of the user. For example, a prefabricated shell is incorporated and reworked or a shell is custom-made using additive manufacturing or manual production.

The orthosis preferably has at least one, preferably two holding straps, which are preferably arranged approximately in the middle of the wearer's upper arm and extend to an upper body strap that surrounds the torso. Said straps serve to reduce/limit the forward or backward swinging of the arm. The length of said straps can be individually adjusted.

Preferably, the tensioning element comprises a strap that is fastened at one end to a first tab, that can be fastened at the second end to the first tab and that is guided through a guide loop on the second of the two tabs. When such a tensioning element is tensioned, the second end, which is not yet fastened to a first tab, is guided through a guide loop. A tensile force can subsequently be applied to this second end of the tensioning element. Said tensile force causes a tensile force on both tabs as, on the one hand, it is transferred to the second tab via the guide loop and transferred to the first tab via the first end of the strap fastened to the first tab. If a sufficient tensile force is applied, the second end of the strap can be fastened to the first tab.

Alternatively or additionally, the tensioning element has a tensioning cord, which is connected to the ventral tab and the dorsal tab and the effective length of which can be altered. Such a tensioning cord may also be provided in the form of a wire. Such a tensioning element preferably has an actuation element by means of which the effective length of the tensioning cord can be altered. Such an element is offered by BOA, for example. In this case, the actuation element is a rotating element, such as a knob that rotates about an axis of rotation. In this case, the tensioning cord is wound up on a spindle or a coil, for example, and its effective length, i.e. especially the distances between the actuation element and its spindle or coil on the one hand and the positions at which the tensioning cord is connected to the respective tab, thus reduced. This results in a tensile force being exerted on the tab and thus on the dimensionally stable shell of the orthosis.

In a preferred embodiment, the tensioning cord is guided through a guide loop on the ventral tab and/or a guide loop on the dorsal tab. This allows for an especially simple fastening and connection between the respective tab and the tensioning cord, which can be made to be especially simple in terms of design. In a preferred embodiment the tensioning cord is guided over at least one deflection pulley, especially preferably over multiple deflection pulleys. This achieves the effect of a pulley so that the required force can also be applied by wearers of the orthosis who have limited motor skills or are physically weak.

In a preferred embodiment, a reinforcement element is arranged on the dimensionally stable shell. This strengthens the dimensionally stable shell. For example, the reinforcement element is designed as a second shell or as at least one strut. The reinforcement element is preferably connected to the first dimensionally stable shell in a non-detachable manner, for example glued or welded on. Alternatively, the reinforcement element is connected to the dimensionally stable shell in a detachable manner, for example by plugging or using a velcro connection. This preferably enables the dimensionally stable shell and the reinforcement element to be individually adapted to the patient. In a preferred embodiment, the dimensionally stable shell is less dimensionally stable than the reinforcement element.

The reinforcement element can also be referred to as a brace and is preferably firmly connected to the dimensionally stable shell. The reinforcement element preferably has a dorsal projection and/or a ventral projection. The dorsal projection extends along the dorsal tab and the ventral projection along the ventral tab. The reinforcement element is preferably designed as a shell. The reinforcement element is preferably arranged on the dimensionally stable shell. This is preferably on the side facing away from the upper arm. Preferably, the reinforcement element is made of a metal, such as aluminium or steel, or a plastic or a fiber-reinforced plastic, such as GRP or CFRP.

Preferably, the tensioning element is fastened directly or indirectly to the reinforcement element, for example via a strap. The tensioning element is preferably fastened directly or indirectly to the two projections of the reinforcement element, for example via a strap.

Preferably, at least one element of an orthopedic rail system is or can be fastened to the dimensionally stable shell and/or the reinforcement element. This is particularly advantageous if, for example, a lower arm is also to be braced, supported or protected by the orthosis. The element of the rail system then extends in the desired direction and can, for example, fix or support a lower arm in a certain and desired position. Preferably, the element of the rail system can be fastened in different positions and/or different orientations on the dimensionally stable shell of the orthosis. This can be achieved by way of a clamp mechanism, a clip or snap-in mechanism, or with screw connections.

Particularly preferably, the rail system is part of the orthosis and comprises at least one body contact element, preferably a lower arm element to be applied to a lower arm and/or a hand element to be applied to a hand. Particularly preferably, the rail system has at least two elements that are positioned connected to each other by means of a joint. Advantageously, the rail system has at least one actuator that is configured to move one element of the rail system relative to another element of the rail system.

Due to the connection of a rail system to the dimensionally stable shell and/or the reinforcement element, forces from an elbow, a lower arm and/or a hand are at least partially, but preferably fully, transferred to the tensioning element. As a result, the load caused by said forces, which may be caused, for example, by the weight of the arm and the orthosis, does not or does not only act on the upper arm or the shoulder joint, but is at least partially, preferably completely, dissipated via the tensioning element. To the extent that it is dissipated, this load does not act as a tensile force on the upper arm, but as a compressive force that acts on the shoulder from above.

The joint of the orthopedic rail system is preferably an elbow joint, which particularly preferably is arranged so that its swivel axis coincides with the swivel axis of the natural elbow of the wearer of the orthosis. Unlike with a textile orthosis, a displacement or twisting of the joint relative to the body part of the wearer of the orthosis is not possible or only to a minor degree. As a result, a position and orientation of the joint, once reached, can be retained for a long time, thereby maintaining the functionality of the orthosis.

This not only applies for elbow joints, but in principle for all elements of the orthopedic rail system that are directly or indirectly arranged on the dimensionally stable shell and/or the reinforcement element. For example, it applies for fixing rails, elbow joints and wrists. They can all be oriented relative to the body part of the wearer of the orthosis and stably kept in this orientation due to the dimensionally stable design of the individual components.

Preferably, a flexible, preferably elastic, padding element is arranged on a side of the dimensionally stable shell facing the upper arm, said padding element preferably being a textile padding element.

The dorsal tab and/or the ventral tab are preferably part of the padding element or are fastened to it, preferably sewn or stuck onto it.

The dimensionally stable shell preferably connects the ventral tab and the dorsal tab on the medial side of the arm. This means that the shell extends between the wearer's arm and torso when the arm is hanging loosely. The opposite side, i.e. the lateral side, is preferably designed to be open. The opening located at this point can be spanned or bridged by one or more straps, but preferably there is no strap.

The joint, for example the elbow joint, is preferably arranged medially on the arm.

This is particularly easy to achieve if the dimensionally stable shell is located medially.

In the following, a number of embodiment examples of the invention will be explained in more detail with the aid of the accompanying figures. They show

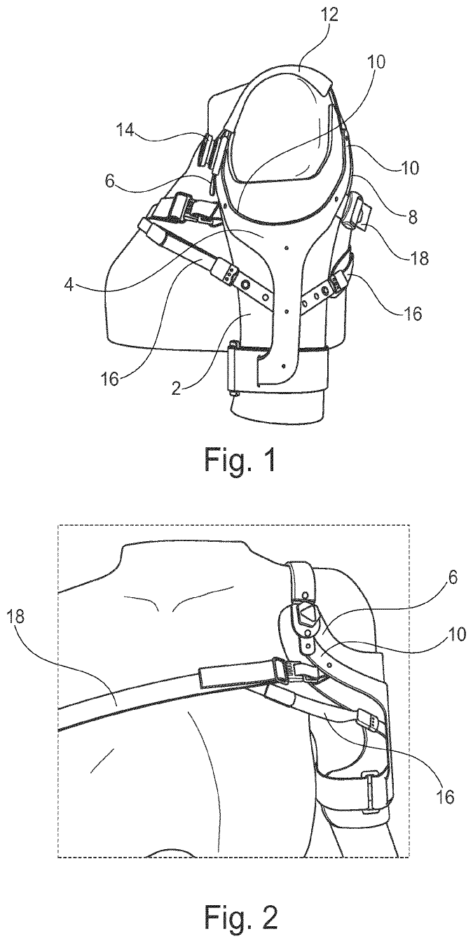

FIGS. 1 and 2—schematic representations of an orthosis according to a first embodiment example of the present invention,

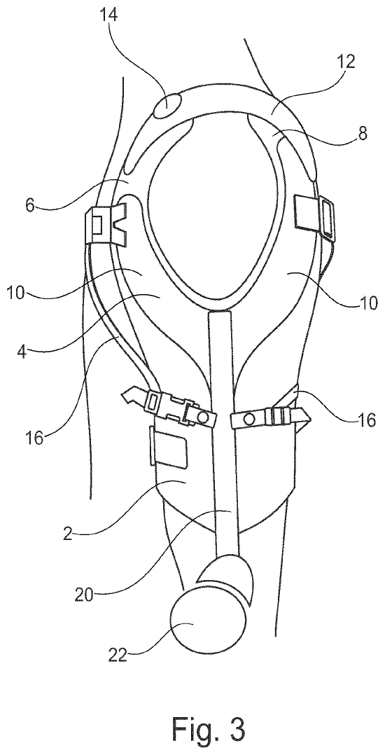

FIG. 3—a schematic representation of an orthosis according to a further embodiment example,

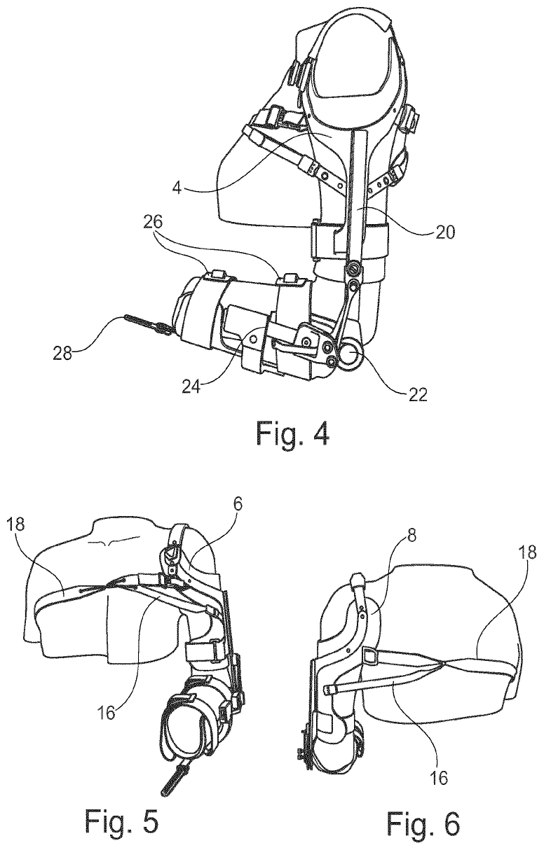

FIGS. 4 to 6—an orthosis according to a further embodiment example of the present invention in different perspectives and

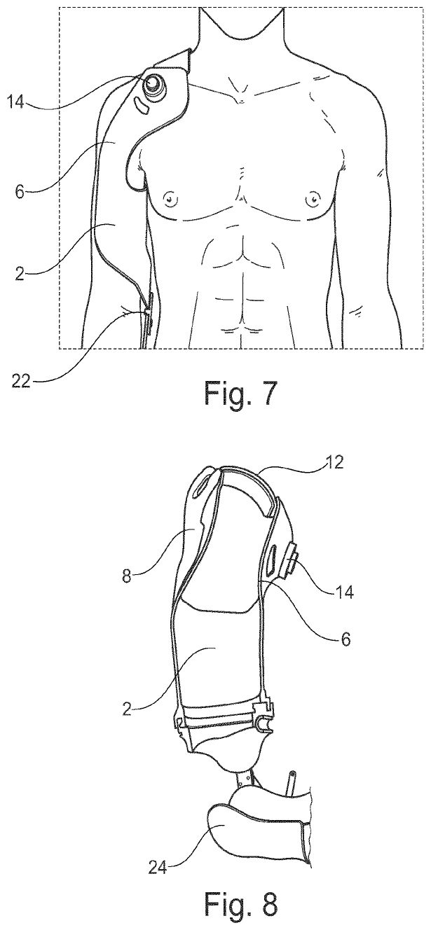

FIGS. 7 and 8—an orthosis according to a further embodiment example of the present invention in the applied state and not-applied state.

FIG. 1 schematically depicts an orthosis according to a first embodiment example of the present invention. It has a dimensionally stable shell 2 on which a reinforcement element 4 is arranged. The dimensionally stable shell has a ventral tab 6 and a dorsal tab 8. The reinforcement element 4 also has two projections 10 which extend along the two tabs 6, 8. Above the shoulder, the two tabs 6, 8 are connected to each other by a strap 12. There is an actuation element 14 on this strap 12, said element being designed as a knob in the embodiment example shown. It is configured to tension a tensioning element, which is not depicted in the figures. Two additional holding straps 16, which connect the dimensionally stable shell 2 to an upper body strap 18, further restrict a range of movement of the upper arm when the orthosis is applied.

FIG. 2 shows a different perspective of the orthosis from FIG. 1. The upper body strap 18, to which the two holding straps 16 are fastened, can now be recognized more clearly. In addition, one can clearly see that the ventral tab 6 extends in the proximal direction, starting from the wearer's upper arm, and extends along said ventral tab 6 of one of the two projections 10 of the reinforcement element 4.

FIG. 3 depicts a side view of another configuration of an orthosis according to the present invention with the reinforcement element 4 that comprises the two projections 10, which extend along the ventral tab 6 and the dorsal tab 8. The two tabs 6, 8 again form part of the dimensionally stable shell 2. The two holding straps 16 are also provided. Above the shoulder, the two tabs 6, 8 are connected to the strap 12 where the actuation element 14 for the tensioning element is located. In the distal area of the dimensionally stable shell, a rail element 20 is arranged on the reinforcement element 4, a joint 22 of a rail system being arranged at the lower end of said rail element.

FIG. 4 depicts a side view of a further configuration of an orthosis according to the present invention. The rail element 20 with the joint 22 arranged thereon is located on the reinforcement element 4. In the embodiment example shown, said joint 22 connects the rail element 20, which extends along the wearer's upper arm when applied, to a lower arm rail 24, which extends along the lower arm. The joint 22 is preferably arranged on the rail element 20 and the lower arm rail 24 in such a way that its swivel axis extends parallel and preferably coaxially to the swivel axis of the wearer's elbow joint. The lower arm rail 24 can be fastened to the lower arm via lower arm straps 26, thereby ensuring that the orthosis is also held firmly and securely on the lower arm. The orthosis is configured, by way of the joint 22 and the rails 20, 24 that it connects, to apply a force to the lower arm, to support, limit and/or train the movement of the wearer's elbow joint and/or to support, relieve and/or protect the elbow joint. A fastening element 28 is arranged on the lower arm rail 24 to which a hand support element, not depicted here, can be arranged. The orthosis illustrated can thus be combined with a known hand orthosis.

FIGS. 5 and 6 show different perspectives of the orthosis from FIG. 4. FIG. 5 shows a frontal view. In this case, the upper body strap 18 placed around the torso and the holding strap 16 that restricts the movement of the upper arm can be seen particularly clearly. The rear view shown in FIG. 6 demonstrates that the upper body strap 18 is placed around the torso. It preferably connects the ventral tab 6 with the dorsal tab 8. In the embodiment example shown, the orthosis also has a holding strap 16 in the backwards area.

FIG. 7 depicts an orthosis according to a further embodiment example of the present invention in the applied state. FIG. 8 shows a lateral view of the same orthosis in the non-applied state. The ventral tab 6 and the dorsal tab 8 are each designed as part of the dimensionally stable shell 2. The shell 2 and the two tabs 6, 8 are produced as components made of carbon fiber composite (CFRP), which lends them sufficient mechanical strength while weighing little themselves. The strap 12 extends over the wearer's shoulder. One can recognize the tensioning element 14, by way of which the effective length of the tensioning cord can be altered. Such an element is offered by BOA, for example. The shell 2 is arranged medially, i.e. it extends between the user's arm and torso. The joint 22 is also positioned medially on the arm.

A lower arm rail 24 is arranged via a joint 22 at the distal end of the dimensionally stable 2, said rail being designed as a CFRP component. The CFRP components are equipped with a padding layer, which is arranged facing the body part.

In FIG. 8 is particular, it is clear that the shell is designed as two parts. It comprises an upper or proximal section and a lower or distal section. Both sections are connected to each other by way of a motion device that connects the two parts so that they can swivel in relation to each other. This renders it possible to swivel a lower arm, on which the lower arm rail is positioned, in a predetermined angular range. As a result, a position of the lower arm relative to the shoulder can be adjusted, for example. The lower arm can be moved either by an external force, such as a movement performed by the user's other arm, or by a movement of its own accord.

### REFERENCE LIST

2 dimensionally stable shell
4 reinforcement element
6 ventral tab
8 dorsal tab
10 projection
12 strap
14 actuation element
16 holding strap
18 upper body strap
20 rail element
22 joint
24 lower arm rail
26 lower arm strap
28 fastening element

1. A method for fastening an orthesis to an upper arm, the orthosis comprising a dimensionally stable shell with a ventral tab and a dorsal tab, wherein the ventral tab and the dorsal tab are connected or connectable by a tensioning element, the method comprising:
applying the dimensionally stable shell to an upper arm,
tensioning the tensioning element so that a tensile force is exerted on the ventral tab and the dorsal tab, said tensile force acting in a proximal direction.

2. The method according to claim 1, wherein the ventral tab and the dorsal tab are connected to each other before the tensioning element is tensioned.

3. The method according to claim 2, wherein the ventral tab and the dorsal tab are connected to each other above a shoulder of the upper arm.

4. An orthosis, comprising:
a dimensionally stable shell with a ventral tab and a dorsal tab; and
a tensioning element for applying a tensile force to the ventral tab and the dorsal tab, wherein the tensile force is adjustable by tensioning the tensioning element, and
wherein the orthosis is configured for fastening to an upper arm according to the method of claim 1.

5. The orthosis according to claim 4, wherein the tensioning element comprises a strap fastened or fastenable at a first end to either the ventral tab or the dorsal tab, and is fastened or fastenable at a second end to either the ventral tab or the dorsal tab, and wherein at least one of the ventral tab and the dorsal tab comprises a guide loop, wherein the orthosis is configured such that the tensioning element is guided through a guide loop when the tensioning element is connected to the ventral tab and the dorsal tab.

6. The orthosis according to claim 4, wherein the tensioning element has a tensioning cord connected to the ventral tab and the dorsal tab, wherein an effective length of the tensioning cord is alterable.

7. The orthosis according to claim 6, wherein the tensioning cord is guided through at least one loop on the ventral tab and/or at least one loop on the dorsal tab.

8. The orthosis according to claim 1 further comprising a reinforcement element arranged on the dimensionally stable shell.

9. The orthosis according to claim 8, wherein the reinforcement element is made of a metal, or a plastic or a fiber-reinforced plastic.

10. The orthosis according to claim 8 wherein the reinforcement element (4) comprises one or more projections arranged on the dorsal a and/or on the ventral tab.

11. The orthosis according to claim 8 wherein the dimensionally stable shell and/or the reinforcement element are configured to have an element of an orthopedic rail system fastened thereto.

12. The orthosis according to claim 4 further comprising an orthopedic rail system fastened to the dimensionally stable shell or a reinforcement element arranged on the dimensionally stable shell, wherein the rail system comprises at least one body contact element configured to be applied to a lower arm and/or a hand.

13. The orthosis according to claim 4 further comprising an orthopedic rail system fastened to the dimensionally stable shell or a reinforcement element arranged on the dimensionally stable shell, wherein the rail system comprises at least two elements that are arranged together by a joint.

14. The orthosis according to claim 4 further comprising:
an orthopedic rail system fastened to the dimensionally stable shell or a reinforcement element arranged on the dimensionally stable shell; and
at least one actuator is arranged on the rail system, said g least one actuator is being configured to move an element of the rail system relative to another element of the rail system.

15. The orthosis according to claim 4, wherein a geometric form of the dimensionally stable shell is adapted to the upper arm.

16. The orthosis according to claim 4 further comprising a flexible, padding element is arranged on a side of the dimensionally stable shell facing the upper arm.

17. The orthosis according to claim 16 wherein said padding element is comprised of a textile.
